(19) United States
(12) Patent Application Publication
(10) Pub. No.: US 2014/0343336 A1
(43) Pub. Date: Nov. 20, 2014

(54) PROCESS FOR THE OXIDATIVE REGENERATION OF A DEACTIVATED CATALYST AND AN APPARATUS THEREFOR

(71) Applicants: SHELL INTERNATIONALE RESEARCH MAATSCHAPPIJ B.V., The Hague (NL); SHELL OIL COMPANY, Houston, TX (US)

(72) Inventors: Sivakumar SADASIVAN VIJAYAKUMARI, Amsterdam (NL); Jeroen VAN WESTRENEN, Amsterdam (NL)

(21) Appl. No.: 14/360,947

(22) PCT Filed: Nov. 28, 2012
(86) PCT No.: PCT/EP2012/073781

§ 371 Date: May 28, 2014

(30) Foreign Application Priority Data
Nov. 28, 2011 (EP) 11190949.5

(51) Int. Cl. B01J 29/90; C07C 4/06; C07C 1/20

(52) U.S. Cl. CPC B01J 29/90; C07C 1/20; C07C 4/06; C07C 2529/00
USPC 585/324; 585/638; 502/34; 585/651; 585/653; 422/233

(57) ABSTRACT

The present invention relates to a process for the oxidative regeneration of a deactivated catalyst comprising providing a catalyst comprising molecular sieve in hydrogen form to a guard zone; passing a regeneration gas stream comprising oxidant through the guard zone to remove part of one or both of any alkali metal ion and alkaline earth metal ion from the regeneration gas stream, to provide a treated regeneration gas stream; providing deactivated catalyst comprising molecular sieve in a regeneration zone, said deactivated catalyst from one or both of an oxygenate to olefin process and an olefin cracking process; regenerating the deactivated catalyst in the regeneration zone with the treated regeneration gas stream to provide regenerated molecular sieve catalyst; wherein said catalyst in said guard zone is one or both of deactivated catalyst comprising molecular sieve in hydrogen form and regenerated catalyst comprising regenerated molecular sieve in hydrogen form.

### FIELD OF THE INVENTION

The present invention relates to a process for the oxidative regeneration of a deactivated catalyst comprising molecular sieve, such as zeolite, specifically deactivated catalyst obtained from one or both of an oxygenate to olefin (OTO) process and an olefin cracking process (OCP), an apparatus therefore and a process for the preparation of olefinic product incorporating such a regeneration process.

### BACKGROUND OF THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted into for instance methanol or dimethyl ether (DME), both of which are suitable feedstocks for an OTO process.

In an OTO process, an oxygenate such as methanol is provided to a reaction zone of a reactor comprising a suitable conversion catalyst whereby it is converted to ethylene and propylene. In addition to the desired ethylene and propylene, a substantial part of the methanol is converted to higher hydrocarbons including C4+ olefins and paraffins. The effluent from the reactor comprising the olefins, any unreacted oxygenates such as alcohols or ethers, particularly methanol and dimethyl ether and other reaction products such as water may then be treated to provide separate component streams. Unreacted oxygenates and water can be separated from the reaction effluent, for instance by contacting with a cooled aqueous stream in a quench zone. In order to increase the ethylene and propylene yield of the process, the C4+ olefins may be recycled to the reaction zone or alternatively further cracked in a dedicated olefin cracking process zone to produce further ethylene and propylene.

International patent application no. PCT/U.S.2005/025666 (WO 2006/023189) discloses a process for regenerating a molecular sieve catalyst. Molecular sieve catalysts used for converting oxygenates to olefins require periodic regeneration in order to maintain catalyst activity. The regeneration can burn off carbonaceous deposits formed on the catalyst during oxygenate conversion and can be carried out in the presence of oxygen.

WO 2006/023189 discloses that the molecular sieve catalysts were found to be sensitive to various contaminants. In particular, contaminants such as sodium chloride, which can be present in the air used in regeneration, were found to cause serious damage to the molecular sieve catalysts. The process of WO 2006/023189 seeks to remove salts and other contaminants from the air entering the regenerator.

One problem with the process of WO 2006/023189 is that it requires a treatment means for the air used to regenerate the catalyst, and the associated CAPEX. The treatment means may cool the regeneration air to condense out excess moisture such that air salinity will be entrained with the condensate, or may be adsorptive means in the form of low pressure drop fixed bed adsorbents or “drying wheels” which can remove both moisture and entrained aerosols of saline particles.

### SUMMARY OF THE INVENTION

The present invention provides an improved process of, and apparatus for regenerating a deactivated catalyst comprising molecular sieve, which does not require the cooling of the regeneration air to remove air salinity with the condensed moisture. Reference herein to a deactivated catalyst comprising molecular sieve is to a catalyst comprising molecular sieve, which has been deactivated at least due to the deposition of carbonaceous matter on the catalyst and in particular on the active sites of the molecular sieve. Thus, OPEX savings are provided by dispensing with the cooling step. This also provides CAPEX savings in terms of the cooling units which are no longer required.

In addition, the present invention provides an improved process which does not require a separate adsorptive means to remove both moisture and entrained aerosols of saline particles, particularly an adsorptive means distinct from the catalyst which is to be regenerated, thus providing OPEX savings in the purchase of the adsorptive means.

The invention treats the regeneration gas stream with a portion of the catalyst comprising molecular sieve in hydrogen form which is to be regenerated or has been regenerated, i.e. the catalyst from the OCP and/or OTO process, in a guard zone to provide a treated regeneration gas stream prior to passing it to the regeneration zone. The treatment in the guard zone can adsorb at least a part of one or more contaminants present. In particular, the molecular sieve in hydrogen form can ion exchange with any alkali metal ion and/or any alkaline earth metal ion in the regeneration gas thereby removing at least a portion of any such ions present from the regeneration gas stream. Spent catalyst comprising ion exchanged molecular sieve is, i.e. molecular sieve not in the hydrogen form, produced. It is not a requirement that all the catalyst comprising molecular sieve in the guard zone is in hydrogen form. It will be apparent that as ion exchange with alkali metal ion and/or alkaline earth metal ion occurs, the proportion of molecular sieve in hydrogen form will decrease. The guard zone provides a treated regeneration gas stream which is depleted in one or both of any alkali metal ion and alkaline earth metal ion compared to an untreated stream comprising such ions.

In addition, the treatment of the regeneration gas stream with the catalyst comprising molecular sieve in hydrogen form may also lower the concentration of other contaminants in the treated regeneration gas compared to the untreated regeneration gas. For instance, the catalyst comprising molecular sieve may also absorb at least a portion of any water present in the regeneration gas. Furthermore, passing the regeneration gas through the guard zone comprising catalyst may also remove at least a portion of any solid particulates present in the regeneration gas, with the solid particulates becoming captured in the guard zone. The solid particulates may be, for instance one or both of dust and soot particles.

Advantageously, the guard zone need not comprise fresh catalyst comprising fresh molecular sieve, but can operate with deactivated catalyst comprising molecular sieve and/or regenerated catalyst comprising regenerated molecular sieve, as long as at least a portion of the molecular sieve is present in the hydrogen form so that the acid sites can ion exchange with one or both of any alkali metal ions and any alkaline earth metal ions present in the regeneration gas.

Typically, the activity of the catalyst in the reaction zone will decline over time through use, which decline cannot be reversed by oxidative regeneration, such that a portion of the catalyst can be removed and replaced with fresh catalyst in order to maintain a desired average catalyst activity. This catalyst removal and replacement may be carried out periodically in a batch process, or constantly in a continuous process. The removal may occur from one or both of the reaction zone and the regeneration zone. The catalyst removed during the catalyst replenishment is ideal and preferred for use in the guard zone.

Over time, the catalyst comprising molecular sieve in the guard zone may become essentially completely ion exchanged with salt, such that it is no longer in the hydrogen form. The completely ion exchanged catalyst. i.e. comprising ion exchanged molecular sieve, also referred to as spent catalyst, can be removed from the guard zone and the guard zone replenished with replacement catalyst comprising molecular sieve in hydrogen form. This catalyst removal and replacement may be carried out periodically in a batch process, or constantly in a continuous process.

It is preferred that the guard zone is maintained such that at least 10 wt. % of the molecular sieve in the catalyst comprising molecular sieve in the guard zone is in hydrogen form, based upon the total weight of molecular sieve present in the guard zone, more preferably at least 15 wt. %, still more preferably at least 25 wt. %.

The guard zone should be separated from the regeneration zone such that there can be no transfer of catalyst from the guard zone to the regeneration zone and/or reaction zone. In this way, ion exchanged catalyst from the guard zone is prevented from contaminating the catalyst to be used in the OCP or OTO process.

In a first aspect, the present invention provides a process for the oxidative regeneration of a deactivated catalyst to provide a regenerated catalyst, said process comprising at least the steps of:
providing catalyst comprising molecular sieve in hydrogen form to a guard zone;
passing a regeneration gas stream comprising oxidant through the guard zone to remove at least a part of one or both of any alkali metal ion and any alkaline earth metal ion from the regeneration gas stream by ion exchange with the molecular sieve of the catalyst, to provide a treated regenerated gas stream comprising oxidant;
providing deactivated catalyst comprising molecular sieve in hydrogen form in a regeneration zone, said deactivated catalyst obtained from one or both of an oxygenate to olefin process and an olefin cracking process, wherein said regeneration zone is separated from said guard zone such that there is no transfer of catalyst from the guard zone to the regeneration zone;
regenerating the deactivated catalyst in the regeneration zone with the treated regeneration gas stream to provide a regenerated catalyst comprising regenerated molecular sieve in hydrogen form;
wherein said catalyst in said guard zone is one or both of deactivated catalyst comprising molecular sieve in hydrogen form and regenerated catalyst comprising regenerated molecular sieve in hydrogen form.

For the avoidance of doubt, it is pointed out that the deactivated catalyst comprising molecular sieve in hydrogen form in the regeneration zone is deactivated catalyst from, i.e. obtained from, one or both of an oxygenate to olefin process and an olefin cracking process. In the present context, the terms “from” and “obtained from” mean that the catalyst has been used in such a process or processes. In oxygenate to olefin processes and olefin cracking processes, the catalyst comprising molecular sieve in the hydrogen form is typically deactivated due to the deposition of carbonaceous matter on the catalyst, in particular on the active sites of the molecular sieve in the catalyst. Therefore preferably the deactivated catalyst further comprises carbonaceous deposits. Such deactivated catalyst may have experienced a decline in catalytic activity through use. Similarly, the catalyst comprising molecular sieve in hydrogen form in the guard zone is one or both of the deactivated catalyst comprising molecular sieve and the regenerated catalyst comprising regenerated molecular sieve from, i.e. obtained from, one or both of an oxygenate to olefin process and an olefin cracking process. Preferably, the catalyst comprising molecular sieve in hydrogen form in the guard zone is regenerated catalyst comprising regenerated molecular sieve in hydrogen form.

It is not essential that the catalyst in the guard zone is identical to the catalyst to be regenerated in the regeneration zone. For instance, the catalyst in the guard zone may be regenerated catalyst, while the regeneration zone may comprise deactivated catalyst.

Similarly, the catalyst in the guard zone may be spent or regenerated catalyst from an OTO process, while the deactivated catalyst in the regeneration zone may be from an olefin cracking process, or vice versa. Thus, when the catalyst in the guard zone is deactivated catalyst, it may be from the same batch or a different batch to that to be regenerated in the regeneration zone.

In one embodiment, the catalyst in the guard zone may be one or both of deactivated catalyst comprising molecular sieve in hydrogen form and regenerated catalyst comprising regenerated molecular sieve in hydrogen form. This may occur, for instance, when a portion of the deactivated catalyst is transferred to the guard zone prior to regeneration, or a portion of the regenerated catalyst is passed to the guard zone from the regeneration zone after regeneration.

In one embodiment, the deactivated catalyst comprising molecular sieve may be provided by one or both of the steps:
reacting an oxygenate feedstock comprising oxygenate in an oxygenate reaction zone in the presence of a catalyst comprising molecular sieve in hydrogen form to produce the deactivated catalyst comprising molecular sieve in hydrogen form and comprising carbonaceous deposits and a reaction effluent stream comprising unreacted oxygenate, olefinic product and water; and
reacting a C4+ hydrocarbon feedstock comprising olefin in an olefin cracking process reaction zone in the presence of a catalyst comprising molecular sieve in hydrogen form to produce the deactivated catalyst comprising molecular sieve in hydrogen form and comprising carbonaceous deposits and a reaction effluent stream comprising unreacted C4+ hydrocarbon and an olefinic product comprising one or both of ethylene and propylene.

In another embodiment, the deactivated catalyst comprising molecular sieve in hydrogen form may be provided by the steps of:
(1) reacting an C4+ hydrocarbon feedstock comprising olefin in an olefin cracking process reaction zone in the presence of a catalyst comprising molecular sieve in hydrogen form to produce the deactivated catalyst comprising molecular sieve in hydrogen form and comprising carbonaceous deposits and a reaction effluent stream comprising unreacted C4+ hydrocarbon and one or both of ethylene and propylene; and
(2) reacting an oxygenate feedstock comprising oxygenate in an oxygenate to olefin reaction zone in the presence of the deactivated catalyst of step (1) to produce deactivated catalyst comprising molecular sieve in hydrogen form and comprising carbonaceous deposits and a reaction effluent stream comprising unreacted oxygenate, olefinic product and water.

In a further embodiment, the step of providing a catalyst comprising molecular sieve in hydrogen form to a guard zone comprises the step of:
passing a portion of deactivated catalyst or regenerated catalyst from the regeneration zone to the guard zone.

In a further embodiment the alkali metal ion may be one or more selected from the group comprising Li³⁰ , Na³⁰ and K⁺, more particularly Na³⁰ . In a still further embodiment, the alkaline earth metal ion may be one or more selected from the group comprising Mg²⁺, Ca²⁺, Sr²⁺ and Ba²⁺, more particularly Ca²⁺. Typically the metal ion comprises one or more of Li⁺, Na⁺, K⁺, Mg²⁺, Ca²⁺ and Sr²⁺, more typically Na⁺ and Ca²⁺.

In another embodiment, one or both of any alkali metal ion and any alkaline earth metal ion may be present as a salt i.e. alkali metal salt or alkaline earth metal salt, such as NaCl and/or CaCl₂. The salt may be selected from one or more of the group comprising sodium chloride, lithium chloride, potassium chloride and calcium chloride.

The salt may be in the form of a solid salt and/or an aqueous solution of the salt. The aqueous salt solution can be adsorbed by the molecular sieve in hydrogen form. The alkali metal ion and/or alkaline earth metal ion may then ion exchange with the hydrogen sites on the molecular sieve. The solid salt particles can be removed from the regeneration gas by capture in the guard zone. Solid NaCl particles may be formed, for instance, by the evaporation of water from saline, such as seawater, while the aqueous solution of NaCl may comprise droplets of seawater.

In a further embodiment, the regeneration gas stream may further comprise water and the treated regeneration gas stream may be a water depleted stream, with at least a portion of any water present being adsorbed by the molecular sieve in the guard zone.

In another embodiment of the process, the regeneration gas stream may comprise one or more of alkali metal ion and alkaline earth metal ion and the treated regeneration gas stream may be depleted in one or more of alkali metal ion and alkaline earth metal ion. The step of passing the regeneration gas stream through the guard zone may further provide a spent catalyst comprising ion exchanged molecular sieve, i.e. a molecular sieve in which a portion of the hydrogen sites have been replaced with one or both of alkali metal ion and alkaline earth metal ion.

In yet another embodiment of the process, the step of passing a regeneration gas stream through the guard zone further provides spent catalyst comprising ion exchanged molecular sieve, and the process further comprises the steps of:
removing spent catalyst comprising ion exchanged molecular sieve from the guard zone;
re-stocking the guard zone with catalyst comprising molecular sieve in hydrogen form.

As used herein, the term “ion exchanged molecular sieve” refers to molecular sieve in which sites in hydrogen form have been ion exchanged with one or both of alkali metal and alkaline earth metal ions.

In a still further embodiment of the process, the steps of removing spent catalyst and re-stocking the guard zone with catalyst are carried out to maintain at least 10 wt. % of the molecular sieve in catalyst comprising molecular sieve in the guard zone is in the hydrogen form, based on the total weight of molecular sieve present in the guard zone, more preferably at least 15 wt. %, still more preferably at least 25 wt. %.

In yet another embodiment of the process, the guard zone and the regeneration zone are independently selected from a fixed bed, a fluid bed and a solid/gas contactor such as a cyclone.

In a still further embodiment of the process, the step of regenerating the deactivated catalyst comprises oxidising the carbonaceous deposits with the treated regenerated gas stream.

In another embodiment, the step of regenerating the deactivated catalyst may be carried out at a regeneration temperature in the range of from 550 to 750° C., more preferably in the range of from 600 to 650° C.

In a further embodiment, one or both of the temperature and mass flow of the treated regeneration gas stream may be controlled to control the rate of the regeneration of the deactivated catalyst in the regeneration gas step. The oxidation of carbonaceous material such as coke is exothermic such that the rate of the reaction can be controlled by manipulating the reaction temperature by controlling one or both of the temperature and the mass flow of the treated regeneration gas.

In a further embodiment, the process may further comprise, prior to passing a regeneration gas stream through the guard zone, the step of:
heating a regeneration gas stream to provide a heated regeneration gas stream;
such that the treated regeneration gas stream is a heated stream.

In another embodiment, the process may further comprise, between the step of passing a regeneration gas stream through the guard zone and the step of regenerating the catalyst, the step of:
heating the treated regeneration gas stream.
   The step of heating the treated regeneration gas stream provides a treated regeneration gas stream as a heated stream.

In a still further embodiment of the process, the oxidant my be oxygen. In another embodiment, the regeneration gas stream may comprise air. Typically the regeneration gas stream is an air stream.

In yet another embodiment of the process, the molecular sieve is selected from the group comprising silicoaluminophosphate and aluminosilicate. The molecular sieve is preferably an aluminosilicate having at least a 10-membered ring zeolite structure. Still more preferably, the molecular sieve comprises one or more of the group comprising a TON-type aluminosilicate, such as ZSM-22, a MTT-type aluminosilicate, such as ZSM-23, and a MFI-type aluminosilicate, such as ZSM-5. The molecular sieve should at least partially be in hydrogen form.

In a second aspect, the present invention provides a process for the preparation of olefinic product, the process comprising at least the steps of:
(a) reacting an oxygenate feedstock comprising oxygenate in an oxygenate to olefin reaction zone in the presence of a OTO catalyst comprising molecular sieve in hydrogen form to produce a deactivated OTO catalyst comprising molecular sieve in hydrogen form and a reaction effluent stream comprising unreacted oxygenate, olefinic product and water;
(b) regenerating the deactivated OTO catalyst according to a process of the first aspect and its embodiments discussed above, wherein the deactivated catalyst comprising molecular sieve in hydrogen form is the deactivated OTO catalyst, to provide a regenerated OTO catalyst.

The olefinic product typically comprises one or more of ethylene, propylene, butylene(s) and pentylene(s). Preferably the olefinic product comprises ethylene and optionally one or more of propylene, butylene(s) and pentylene(s).

In a third aspect, the present invention provides a process for the preparation of olefinic product comprising one or both of ethylene and propylene, the process comprising at least the steps of:
(a) reacting an C4+ hydrocarbon feedstock comprising olefin in an olefin cracking process reaction zone in the presence of a catalyst comprising molecular sieve in hydrogen form to produce a deactivated catalyst comprising molecular sieve in hydrogen form and a reaction effluent stream comprising unreacted C4+ hydrocarbon and one or both of ethylene and propylene;
(b) regenerating the deactivated catalyst according to a process of the first aspect and its embodiments discussed above to provide a regenerated catalyst.

In a fourth aspect, the present invention provides a process for the preparation of olefinic product, the process comprising at least the steps of:
(a) (1) reacting an C4+hydrocarbon feedstock comprising olefins in an olefin cracking process reaction zone in the presence of a catalyst comprising molecular sieve in hydrogen form to produce a deactivated catalyst comprising molecular sieve in hydrogen form and a reaction effluent stream comprising unreacted C4+ hydrocarbon and one or both of ethylene and propylene;
(2) reacting an oxygenate feedstock comprising oxygenate in an oxygenate to olefin reaction zone in the presence of the deactivated catalyst of step (1) to produce deactivated catalyst comprising molecular sieve in hydrogen form and a reaction effluent stream comprising unreacted oxygenate, olefinic product and water
(b) regenerating the deactivated catalyst of step (2) according to a process of the first aspect and its embodiments discussed above to provide a regenerated catalyst.

In oxygenate to olefin processes and olefin cracking processes, the catalyst comprising molecular sieve in the hydrogen form is typically deactivated due to the deposition of carbonaceous matter on the catalyst, in particular on the active sites of the molecular sieve in the catalyst. Therefore preferably the deactivated catalyst further comprises carbonaceous deposits.

In one embodiment of the third or fourth aspects, the molecular sieve is aluminosilicate molecular sieve, and more particularly zeolite molecular sieve.

In one embodiment of the second, third or fourth aspects, the process further comprises the steps of:
(c) repeating step (a) at least once with the regenerated catalyst;
(d) repeating step (b) at least once with the deactivated catalyst.

In a fifth aspect, the present invention provides an apparatus for the oxidative regeneration of a deactivated catalyst to provide a regenerated catalyst, said apparatus comprising at least:
a guard zone comprising one or both of deactivated catalyst comprising molecular sieve in hydrogen form and regenerated catalyst comprising regenerated molecular sieve in hydrogen form, said spent and regenerated catalyst from one or both of an oxygenate to olefin process and an olefin cracking process, said guard zone having a first inlet for a regeneration gas stream and a first outlet for a treated regeneration gas stream in fluid communication with a first inlet of a regeneration zone;
a regeneration zone comprising deactivated catalyst comprising molecular sieve in hydrogen form, said deactivated catalyst from one or both of an oxygenate to olefin process and an olefin cracking process, said regeneration zone having a first inlet for the treated regeneration gas stream and a first outlet for a regeneration effluent stream.

In one embodiment of the fifth aspect, the regeneration zone may be an OTO or OCP reaction zone, particularly an OTO or OCP reactor.

In another embodiment of the fifth aspect, the regeneration zone may further comprise a second outlet for a fluidised catalyst supply stream, said second outlet in fluid communication with a second inlet of the guard zone.

In a further embodiment of the fifth aspect, the regeneration zone may further comprise a third outlet for a fluidised regenerated catalyst stream, said third outlet in fluid communication with an OTO or OCP reaction zone, and a second inlet for a fluidised deactivated catalyst stream in fluid communication with a first outlet of a reaction zone, and
a OTO or OCP reaction zone having a first inlet for the fluidised regenerated catalyst stream, a first outlet for fluidised deactivated catalyst stream, a second outlet for a reaction effluent stream and a second inlet for a feedstock stream.

In a further embodiment of the fifth aspect, the reaction zone may be a reactor, such as a fluidised bed or riser reactor.

### DETAILED DESCRIPTION OF THE INVENTION

Embodiments of the present invention will now be described by way of example only and with reference to the accompanying non-limited drawing in which:

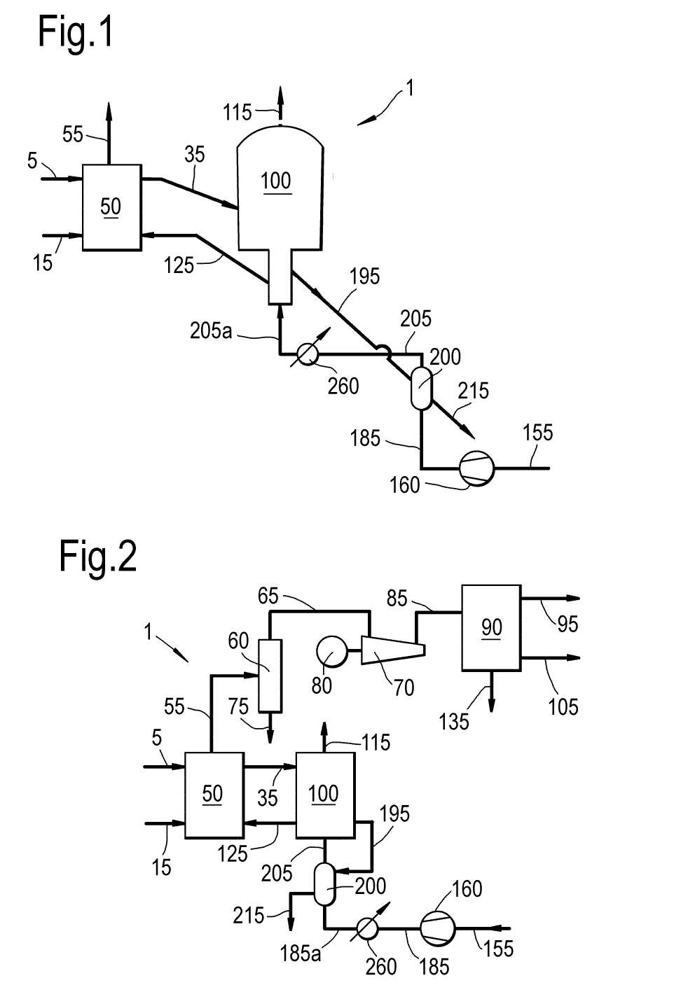

FIG. 1 is a diagrammatic scheme of one embodiment of a process for the oxidative regeneration of a deactivated catalyst described herein.

FIG. 2 is a diagrammatic scheme of another embodiment of a process for the oxidative regeneration of a deactivated catalyst described herein.

The deactivated catalyst regenerated in the process and apparatus described herein can be produced during the catalytic conversion of an oxygenate feedstock to olefinic products in an oxygenate-to-olefins process or during the catalytic cracking of a C4+ hydrocarbon feedstock comprising olefin in an olefin cracking process.

For instance, FIG. 1 shows an apparatus 1 in which the process described herein may be utilised. An oxygenate feedstock stream 5 can be contacted in an OTO reaction zone 50, such as an OTO reactor, with a catalyst for oxygenate conversion under oxygenate conversion conditions, to obtain a reaction effluent comprising olefins, particularly lower olefins. The reaction effluent can be removed from reaction zone 50 as reaction effluent stream 55. Reaction effluent stream 55 may comprise unreacted oxygenate, olefinic product and water and can be processed in a variety of ways known in the art. The embodiment of FIG. 2 discussed below shows one preferred line-up for the processing of the reaction effluent.

Reference herein to an oxygenate feedstock is to an oxygenate-comprising feedstock. In the OTO reaction zone 50, at least part of the feedstock is converted into a product containing one or more olefins, preferably including lower olefins, in particular ethylene and typically propylene.

The oxygenate used in the process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethylether, methylethylether. Preferably, the oxygenate is methanol or dimethylether, or a mixture thereof.

Preferably the oxygenate feedstock comprises at least 50 wt % of oxygenate, in particular methanol and/or dimethylether, based on total hydrocarbons, more preferably at least 70 wt %.

The oxygenate feedstock can comprise an amount of diluent, such as water or steam. In one embodiment, the molar ratio of oxygenate to diluent is between 10:1 and 1:10, preferably between 4:1 and 1:2, in particular when the oxygenate is methanol and the diluent is water (typically steam).

Preferably, in addition to the oxygenate, an olefinic co-feed is provided along with and/or as part of the oxygenate feedstock. FIG. 1 shows the co-feed being supplied to OTO reaction zone 50 as an olefinic co-feed stream 15. Reference herein to an olefinic co-feed is to an olefin-comprising co-feed. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon species.

Preferably, at least 70 wt % of the olefinic co-feed, during normal operation, is formed by a recycle stream of a C4+ hydrocarbon fraction from the OTO reaction effluent. Preferably at least 90 wt % of olefinic co-feed, based on the whole olefinic co-feed, is formed by such recycle stream. In order to maximize production of ethylene and propylene, it is desirable to maximize the recycle of C4 olefins in the effluent of the OTO process. This can be done by recycling at least part of the C4+ hydrocarbon fraction, preferably C4-C5 hydrocarbon fraction, more preferably C4 hydrocarbon fraction, in the OTO effluent. However, a certain part thereof, such as between 1 and 5 wt %, can be withdrawn as purge, since otherwise saturated hydrocarbons, in particular C4s (butane) would build up in the process, which are substantially not converted under the OTO reaction conditions.

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

A variety of OTO processes are known for converting oxygenates, such as for instance methanol or dimethylether to an olefin-containing product, as already referred to above. One such process is described in WO-A 2006/020083. Processes integrating the production of oxygenates from synthesis gas and their conversion to light olefins are described in U.S.20070203380A1 and U.S.20070155999A1.

Catalysts suitable for converting the oxygenate feedstock comprise molecular sieve. Such molecular sieve-comprising catalysts typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanides of the Periodic Table of Elements. Preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, and the FER type. Other suitable zeolites are for example zeolites of the STF-type, such as SSZ-35, the SFF type, such as SSZ-44 and the EU-2 type, such as ZSM-48.

Aluminosilicate-comprising catalyst, and in particular zeolite-comprising catalyst are preferred when an olefinic co-feed is fed to the oxygenate conversion zone together with oxygenate, for increased production of ethylene and propylene.

Preferred catalysts comprise a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a silica-to-alumina ratio, SAR, of at least 60, preferably at least 80. Preferred catalysts may include catalysts comprising one or more zeolites having one-dimensional 10-membered ring channels, i.e. one-dimensional 10-membered ring channels, which are not intersected by other channels. Preferred examples are zeolites of the MTT and/or TON type. In a particularly embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

The molecular sieves in the catalyst are at least partially in the in the hydrogen form, e.g. HZSM-22, HZSM-23, and HZSM-48, HZSM-5. Preferably, at least 50% w/w, more preferably at least 90% w/w, still more preferably at least 95% w/w and most preferably 100% of the total amount of molecular sieve used is in the hydrogen form. It is well known in the art how to produce such molecular sieves in the hydrogen form.

The reaction conditions of the oxygenate conversion, particularly step (a) of the second aspect and step (a)(2) of the fourth aspect, include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Preferably, the oxygenate feedstock is preheated to a temperature in the range of from 200 to 550° C., more preferably 250 to 500° C. prior to contacting with the molecular sieve-comprising catalyst.

The catalyst particles used in the process can have any shape known to the skilled person to be suitable for this purpose, and can be present in the form of spray dried catalyst particles, spheres, tablets, rings, extrudates, etc. Extruded catalysts can be applied in various shapes, such as, cylinders and trilobes. Spray-dried particles allowing use in a fluidized bed or riser reactor system are preferred. Spherical particles are normally obtained by spray drying. Preferably the average particle size is in the range of 1-200 μm, preferably 50-100 μm, still more preferably approximately 60-80 μm. Although the C4+ hydrocarbon fraction in the reaction effluent may be recycled as an olefinic co-feed (e.g. stream 15), in an alternative embodiment not shown in FIG. 1, at least part of the olefins in the C4+ hydrocarbon fraction are converted to ethylene and/or propylene by contacting the C4+ hydrocarbon fraction in a separate unit with a molecular sieve-comprising catalyst, particularly a zeolite-comprising catalyst. This is particularly preferred where molecular sieve-comprising catalyst in the OTO process comprises a least one SAPO, AlPO, or MeAlPO type molecular sieve, preferably SAPO-34. These catalysts are less suitable for converting olefins. Preferably, the C4+ hydrocarbon fraction, particularly in step (a) of the third aspect and step (a)(1) of the fourth aspect is contacted with the zeolite-comprising catalyst at a reaction temperature of 350 to 1000° C., preferably from 375 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar). Optionally, the stream comprising C4+ olefins also contains a diluent. Examples of suitable diluents include, but are not limited to, liquid water or steam, nitrogen, argon, paraffins and methane. Under these conditions, at least part of the olefins in the C4+ hydrocarbon fraction are converted to further ethylene and/or propylene. The further ethylene and/or propylene may be combined with the further ethylene and/or propylene obtained directly from the OTO reaction zone. Such a separate process step directed at converting C4+ olefins to ethylene and propylene is also referred to as an olefin cracking process (OCP).

Catalysts comprising molecular sieve, particularly aluminosilicate-comprising catalysts, and more particularly zeolite-comprising catalysts, have the further advantage that in addition to the conversion of methanol or ethanol, these catalysts also induce the conversion of olefins to ethylene and/or propylene. Therefore, aluminosilicate-comprising catalysts, and in particular zeolite-comprising catalysts, are particularly suitable for use as the catalyst in an OCP. The preferences provided herein above for the oxygenate to olefins catalyst apply mutatis mutandis for the OCP catalyst with the primary exception that the OCP catalyst always comprises at least one zeolite.

Particular preferred catalysts for the OCP reaction, i.e. converting part of the olefinic product, and preferably part of the C4+ hydrocarbon fraction of the olefinic product including olefins, are catalysts comprising at least one zeolite selected from MFI, MEL, TON and MTT type zeolites, more preferably at least one of ZSM-5, ZSM-11, ZSM-22 and ZSM-23 zeolites.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus.

The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

Preferably, the oxygenate to olefins catalyst and the olefin cracking catslyst are the same zeolite comprising catalyst.

Both the OTO process and the OCP may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

In a further embodiment not shown in FIG. 1, catalyst comprising molecular sieve in hydrogen form may be first used in an OCP reaction zone for the conversion of the C4+ olefins of the C4+ hydrocarbon fraction, and subsequently transferred to the OTO reaction zone for conversion of the oxygenate feedstock stream 5 and olefinic co-feed stream 15.

As discussed above, the catalyst can deactivate in the course of the OCP and OTO process to produce deactivated catalyst comprising molecular. The deactivation occurs primarily due to deposition of carbonaceous deposits, such as coke, on the catalyst by side reactions, to produce molecular sieve comprising carbonaceous deposits. The carbonaceous deposits can block the access of the oxygenate feedstock to the active sites of the molecular sieve.

The deactivated catalyst can be regenerated to remove a portion of the carbonaceous deposit such as coke. It is not necessary, and indeed may be undesirable, to remove all the carbonaceous deposit from the catalyst as it is believed that a small amount of residual carbonaceous deposit such as coke may enhance the catalyst performance. Additionally, it is believed that complete removal of the carbonaceous deposit may also lead to degradation of the molecular sieve.

The same catalyst may be used for both the OTO process and OCP. In such a situation, the catalyst comprising molecular sieve, particularly comprising aluminosilicate molecular sieve and more particularly comprising zeolite, may be first used in the OCP. The deactivated catalyst from the OCP may then be used, typically without oxidative regeneration, in the OTO process. The deactivated catalyst from the OTO process may then be regenerated as described herein, and the regenerated catalyst then used again in the OCP.

This line-up may be beneficial because it provides good heat integration between the OCP, OTO and oxidative regeneration processes. The OCP is endothermic and at least a portion of the heat of reaction can be provided by passing catalyst from the regeneration zone to the OCP reaction zone, because the regeneration reaction which oxidizes the carbonaceous deposits from the deactivated catalyst is exothermic.

In the process described herein, the deactivated catalyst comprising molecular sieve comprising carbonaceous deposits can be regenerated with a treated regeneration gas stream 205. In the embodiment shown in FIG. 1, deactivated catalyst is transferred from the reaction zone 50 to a regeneration zone 100, for instance as a fluidised deactivated catalyst stream 35.

The regeneration zone 100 may be a regenerator such as a fluidized bed or moving bed, e.g. a fast fluidized bed or a riser reactor system. Oxidative regeneration may also be carried out in the reaction zone, particularly the reactor itself, such as in a fixed bed reactor or a tubular reactor. Oxidative regeneration of the deactivated catalyst comprising molecular sieve comprising carbonaceous deposits is carried out with a treated regeneration gas stream 205. The treated regeneration gas stream 205 can comprise an oxidant, such as oxygen. Typically, the stream may comprise treated air. The stream has been treated in order to reduce the concentration of one or more of any contaminants, particularly any alkali metal ions and/or any alkaline earth metal ions present and optionally any water.

Preferably the stream has been treated to reduce the concentration of water and one or both of alkali metal ions and alkaline earth metal ions present because the contaminant ions are commonly present in aqueous solution, for instance in the form of salts of the ions dissolved in water droplets.

Alkali metal ions and alkaline earth metal ions can ion exchange with a molecular sieve during oxidative regeneration, particularly a molecular sieve in hydrogen form, such as typical catalysts for OCP and OTO processes, resulting in the deactivation of catalytic activity by the removal of active sites, particularly acidic sites. The loss of active sites on the molecular sieve leads to a decrease in the catalytic activity of the regenerated catalyst comprising the regenerated molecular sieve i.e. oxidative regeneration cannot reverse the deactivation of active sites in hydrogen form by ion exchange. For this reason, catalyst comprising ion exchanged molecular sieve is referred to herein as spent catalyst. Water present in the regeneration gas may hydrothermally damage the molecular sieve comprising carbonaceous deposits at the regeneration temperature.

Thus, the treated regeneration gas stream 205 should be an alkali metal ion and/or alkaline earth metal ion depleted treated regeneration gas stream, which is optionally also water depleted. In the present context, the term “depleted” means that the concentration of the depleted component is less than that of the corresponding untreated stream. Thus, a water, alkali metal ion and alkaline earth metal ion depleted treated regeneration gas stream would have reduced concentrations of water, alkali metal ion and alkaline earth metal ion compared to the stream prior to treatment (i.e. regeneration gas stream 185 in the embodiment of FIG. 1).

In a preferred embodiment, the treated regeneration gas stream 205 comprises less than 500 wt ppb total alkali metal ion and alkaline earth metal ion, more preferably, the less than 300 wt ppb total, still more preferably less than 100 wt ppb total based on the treated regeneration gas stream 205. In another preferred embodiment, the treated regeneration gas stream 205 comprises less than 250 wt ppb sodium ion, more preferably less than 150 wt ppb, still more preferably less than 50 wt ppb based on the treated regeneration gas stream 205. In yet another preferred embodiment, the treated generated gas stream 205 comprises less than 4 mol % water, more preferably less than 3 mol %, still more preferably less than 2 mol % based on the treated regeneration gas stream 205.

The regeneration process can heat the molecular sieve comprising carbonaceous deposits in an oxidising environment supplied by the treated regeneration gas stream. The regeneration step can oxidise the carbonaceous deposits on the molecular sieve comprising carbonaceous deposits to produce gaseous oxides of carbon, which can leave the regeneration zone 100 as regeneration effluent stream 115. The regeneration effluent stream 115 may comprise one or more oxidation products of the carbonaceous material, such as carbon monoxide and carbon dioxide, as well as any unreacted components of the treated regeneration gas stream, such as any unreacted oxidant, and any inert components, such as nitrogen and/or carbon dioxide already present in the treated regeneration gas stream 205 e.g. if air is used.

The oxidative regeneration step can be carried out at a regeneration temperature in the range of from 550 to 750° C., more preferably in the range of from 600 to 650° C. The oxidative regeneration step can be initiated by heating the regeneration zone 100 to regeneration temperature in the presence of the treated regeneration gas stream 205. This can be achieved, for instance, by pre-heating the treated regeneration gas stream 205, such that it is provided as a heated stream, or by pre-heating the corresponding untreated regeneration gas stream 185, such as to a temperature at or above 550° C., to initiate oxidation of the carbonaceous material on the molecular sieve comprising carbonaceous deposits.

In the embodiment of FIG. 1, the treated regeneration gas stream 205 is passed through a regeneration gas heat exchanger 260, such as a heater, where its temperature can be increased to provide regeneration gas stream 205a as a heated stream. In this way, heated treated regeneration gas stream 205a can be used to initiate oxidative regeneration of the deactivated catalyst in regeneration zone 100 after it has been passed through guard zone 200.

In addition, heat exchanger 260 can control the temperature of the heated treated regeneration gas stream 205a to control the rate of the catalyst regeneration in regeneration zone 100.

In an alternative embodiment not shown in FIG. 1, the heat exchanger 260 may be placed downstream of the guard zone 200, for instance to heat the regeneration gas stream 185. This embodiment is described in the embodiment of FIG. 2. However, the configuration of the embodiment of FIG. 1 is preferred because the temperature of the regeneration gas stream 185 passed to the guard zone 200 does not require to be manipulated to control the regeneration reaction, allowing the guard zone 200 to operate at a wider range of temperatures, particularly temperatures lower than that required to support regeneration.

Once the oxidative regeneration reaction is underway, the regeneration gas stream 205 may be heated to a temperature of approximately 250° C. to 300° C., more typically approximately 250° C., to maintain the reaction. Alternatively or additionally, the regeneration zone 100 itself may be heated, for instance by heating elements.

The oxidation of carbonaceous material such as coke which occurs during regeneration is exothermic such that controlling one or both of the temperature and the mass flow of the treated regeneration gas stream 205 can be used to control the regeneration temperature, and therefore rate of the regeneration reaction.

Typically the mass flow of the treated regeneration gas stream 205 is adjusted to achieve a desired reduction in the level of carbonaceous material, such as coke, on the molecular sieve. This reduction is known as the “delta coke level”. The delta coke level is proportional to the heat that is generated by the exothermic oxidative regeneration reaction. The oxidative regeneration step may be carried out in a “full burn” mode in which substantially all the carbonaceous deposits are removed by conversion into carbon dioxide. Alternatively, the oxidative regeneration step may be carried out in “half burn” mode in which more carbonaceous deposit remains on the molecular sieve after regeneration, and mostly carbon monoxide is produced. These two oxidative regeneration modes result in different changes in enthalpy and therefore different changes in temperature. For a given oxidative regeneration mode, the change in temperature can therefore be used as a measure of the extent of coke removal.

It will be apparent that the oxidative regeneration reaction can be terminated by lowering the temperature of the heated treated regeneration gas stream 205a, for instance to a temperature below 250° C., more preferably below 200° C., still more preferably below 150° C., such that the temperature of the molecular sieve in the regeneration zone 100 is reduced below that required to support combustion of the carbonaceous material.

Once the molecular sieve has been heated for a sufficient time to remove at least a portion of the carbonaceous material, the regeneration reaction can be terminated and the regenerated catalyst comprising molecular sieve can be returned to the reaction zone 50, for instance as fluidised regenerated catalyst stream 125. In this way, batch wise or continuous oxidative regeneration processes may be started-up and shut.

However, in a continuous oxidative regeneration process, such as that shown in the embodiment of FIG. 1, the regeneration zone may be continuously supplied with deactivated catalyst from the reaction zone 50 via fluidised deactivated catalyst stream 35. Similarly, fluidised regenerated catalyst stream 125 may be continuously returned to the reaction zone 50. The regeneration zone 100 can be operated at a pre-selected level of delta coke with a balanced mass flow of incoming and outgoing catalyst via fluidised catalyst streams 35 and 125. The regeneration temperature can be controlled by adjusting the mass flow of regeneration gas, particularly treated regeneration gas stream 205 and/or heated treated regeneration gas stream 205a provided as discussed below.

The treated regeneration gas stream 205 is formed by passing regeneration gas stream 185 through a guard zone 200. The regeneration gas stream 185 comprises an oxidant. The oxidant may be one or more of the group selected from oxygen, ozone, oxides of sulphur (such as sulphur trioxide), and oxides of nitrogen, NOₓ (such as N₂O, NO, NO₂ and/or N₂O₅). Oxygen is preferred, such that the regeneration gas stream 185 may comprise air. The air may be diluted with a diluent such as nitrogen or carbon dioxide, if required. It is also advantageous that the regeneration gas stream 185 is substantially free of the aforementioned oxides of nitrogen or oxides of sulphur, in order to avoid their contamination of the regeneration effluent stream 115. A regeneration effluent stream 115 comprising one or both of oxides of nitrogen and oxides of sulphur may require further treatment to remove such pollutants.

The regeneration gas stream 185 may comprise contaminants, some of which, like alkali metal ions, alkaline earth metal ions and water, may be damaging to the molecular sieve to be regenerated. Thus, in this context, alkali metal ions, alkaline earth metal ions and optionally water can all be viewed as contaminants which can be detrimental to the activity of the catalyst i.e. catalyst poisons. These contaminants may occur in the regeneration gas stream 185 if, for instance, the regeneration zone 100 is located in a costal environment, such that regeneration gas comprising air may be contaminated with saline water or solid particulates comprising salt (NaCl).

The concentration of any such contaminants in the regeneration gas can be reduced by passing regeneration gas stream 185 through a guard zone 200 comprising catalyst comprising molecular sieve in hydrogen form. The catalyst comprising molecular sieve in hydrogen form is one or both of deactivated catalyst comprising molecular sieve in hydrogen form and regenerated catalyst comprising regenerated molecular sieve in hydrogen form. The spent or regenerated catalyst is obtained from one or both of the OCP or OTO process i.e. the deactivated catalyst to be regenerated in the regeneration zone 100 or the product of the oxidative regeneration of such a deactivated catalyst (i.e. regenerated catalyst). In the embodiment of FIG. 1, the catalyst is supplied to the guard zone 200 from the regeneration zone 100 as guard zone fluidised catalyst supply steam 195.

The guard zone 200 can facilitate the intimate contact of the heated regeneration gas stream 185a with the catalyst comprising molecular sieve in hydrogen form contained therein, thereby allowing the removal of at least a portion of any contaminants present such as one or more of alkali metal ion, alkaline earth metal ion and water to provide treated regeneration gas stream 205. Any alkali metal and/or any alkaline earth metal ions present in the heated regeneration gas may ion exchange with the hydrogen sites of the molecular sieve, while water may be adsorbed by the molecular sieve. The guard zone 200 may be a fixed bed, a fluidised bed and a solid/gas contactor such as a cyclone.

The guard zone 200 may also capture alkali metal ions and alkaline earth metal ions present in the heated regeneration gas in particulate form, for instance when they are present as entrained solid salts. Other particulate materials, such as dust and soot may also be captured by the guard zone 200.

It will be apparent that removal of one or both of any alkali metal ions and any alkaline earth metal ions by ion exchange with the molecular sieve in hydrogen form will produce ion exchanged molecular sieve in which the acidic hydrogen sites have been replaced with alkali metal ions and/or alkaline earth metal ions. As the acidic sites of the molecular sieve are consumed, the ability of the molecular sieve to remove further alkali metal ions or alkaline earth metal ions will decrease until a spent catalyst comprising ion exchanged molecular sieve is formed. Thus, the process described herein uses a portion of the spent or regenerated catalyst in guard zone 200 in a sacrificial manner, in order to minimise deactivation of the catalyst in the regeneration zone 100 by ion exchange with one or both of alkali metal ions and alkaline earth metal ions.

The spent catalyst can be removed from the guard zone 200 as spent catalyst fluidised stream 215. It will be apparent that the spent catalyst fluidised stream 215, as well as comprising spent catalyst comprising ion exchanged molecular sieve, may further comprise captured particulate solids such as solid NaCl and/or soot. In this way, the spent catalyst fluidised stream 215 can operate to prevent the accumulation of particulate solids in the guard zone 200.

The spent catalyst removed from guard zone 200 by spent catalyst fluidised stream 215 can be treated by means known in the art, for instance with an aqueous solution of ammonium, to ion exchange the deactivated sites comprising one or both of alkali metal and alkaline earth metal ions to the ammonium form followed by calcination to remove ammonia and supply the hydrogen form for re-use.

The guard zone 200 can be replenished with molecular sieve in hydrogen form by guard zone fluidised catalyst supply steam 195, for instance from the regeneration zone 100. This removal and re-stocking of the guard zone 200 can be carried out batch-wise or continuously.

The spent catalyst from the guard zone 200 should not be returned to the regeneration zone 100 or reaction zone 50 because the ion exchange of the hydrogen sites of the molecular sieve with one or both of alkali metal ion and alkaline earth metal ion leads to catalyst deactivation.

Consequently, the guard zone 200 must be separated from the regeneration zone 100 and reaction zone 50 in order to prevent contamination of the spent or regenerated catalyst with spent catalyst. Thus, although spent or regenerated catalyst may be provided to the guard zone 200 from the regeneration zone 100 or elsewhere, such as reaction zone 50, spent catalyst should not pass from the guard zone 200 to the regeneration zone 100 or reaction zone 50.

In an alternative embodiment not shown in FIG. 1, the regeneration zone 100 and guard zone 200 may be present in the same shell, but segregated by a gaseous permeable and catalyst impermeable separator 150, such as a mesh comprising pores of sufficiently small size to prevent passage of the catalyst between zones. In such an embodiment, the regeneration and guard zones 100, 200 would be fixed bed reactors.

In an embodiment not shown in FIG. 1, the regeneration gas may be passed through a particulate removal means. Such a filtration step can remove solid particulates such as those comprising dust and/or salt (NaCl) from the regeneration gas before it is passed through guard zone 200, thereby preventing them from accumulating in the zone. For instance, the particulate removal means may be provided in regeneration gas stream 185 or regeneration gas feed stream 155 discussed below.

Typically, the particulate removal means can remove particles with a particle size of greater than 30 micrometers, more typically greater than 10 micrometers. The particulate removal means may be one or more selected from the group comprising screens or meshes, inertial separators, viscous impingement filters and barrier filters.

The regeneration gas stream 185 may be provided as a pressurised stream, for instance by a pressurising means 160 such as the blower shown in FIG. 1 or alternatively a compressor. The pressurising means should provide sufficient pressure to overcome any pressure drop in guard zone 200 and pass the treated regeneration gas to the regeneration zone 100. Typically the regeneration zone 100 will operate at a pressure of approximately 1 bar.

If the pressurising means 160 provides the regeneration gas stream 185 at a temperature above that desirable for downstream equipment or steps, it may be cooled by systems known in the art, such as an air or water cooler. The pressurising means 160 may be fed at its suction by a regeneration gas feed stream 155, which may comprise air or another suitable oxidant-comprising stream.

FIG. 2 provides an alternative embodiment of the process and apparatus described herein. Identical reference numerals to the embodiment of FIG. 1 correspond to identical streams or equipment.

In contrast to the embodiment of FIG. 1, FIG. 2 shows a line-up in which the regeneration gas stream 185 is passed through a regeneration gas heat exchanger 260, such as a heater, where its temperature can be increased to provide regeneration gas stream 185a as a heated stream. In this way, heated regeneration gas stream 185a can be used to control rate of the catalyst regeneration of the deactivated catalyst in regeneration zone 100 after it has been passed through guard zone 200.

FIG. 2 additionally shows one embodiment for the processing of reaction effluent stream 55 from the reaction zone 50. This embodiment is also applicable to the embodiment shown in FIG. 1. The olefinic product obtained from an OTO process can comprise ethylene and/or propylene, which may be separated from the remainder of the components in the reaction effluent stream 55. For instance, the reaction effluent stream 55 can be passed to quench zone 60, where it is separated into a water rich stream 75 comprising oxygenate and a water depleted effluent stream 65 comprising olefinic product. The separation in the quench zone 60 can be effected by contacting the reaction effluent stream 55 with an aqueous quench stream (not shown), such as a water stream, particularly a cooled water stream.

The water depleted effluent stream 65 can then be passed to an effluent compressor 70, in which the pressure of the stream is increased to provide a compressed effluent stream 85 comprising olefinic product. The effluent compressor 70 may be driven by an effluent compressor driver 80, such as an electric motor or a turbine, particularly a steam turbine. The compressed effluent stream 85 may be provided at a pressure above 5 bara, typically above 25 bara, more typically in the range of from 35 to 45 bara.

In an embodiment not shown in FIG. 2, the water depleted effluent stream 65 may be treated to remove any carbon dioxide present, for example, prior to compression or in between compression stages if multiple stages of compression as used. For instance, the water depleted effluent stream 65 may be contacted with an aqueous alkaline stream, such as a stream comprising an alkali metal hydroxide, to absorb the acid-forming carbon dioxide gas.

Gas-liquid separators (not shown), such as a knock-out drums, for the removal of any condensed phase such as water and C5+ hydrocarbons may be present after compression, or after each stage of compression if a multi-stage compression system is used. Subsequently, the compressed effluent stream 85 can be passed to an olefinic separation zone 90, such as a distillation zone, preferably a cryogenic distillation zone, to provide two or more olefinic component streams 95, 105, 135.

The olefinic product preferably comprises two or more of the group selected from ethylene, propylene, butylene(s), pentylene(s) and hexylene(s). Consequently, each of the two or more olefinic component stream may comprise at least one of the group selected from ethylene, propylene, butylene(s), pentylene(s) and hexylene(s). In the embodiment of FIG. 2, olefinic separation zone 90 may comprise a deethaniser providing a first olefinic component stream 95 comprising ethylene, a depropaniser providing a second olefinic component stream 105 comprising propylene and a third olefinic component stream 135 comprising C4+ olefins such as one or more of butylene(s), pentylene(s) and hexylene(s).

In a preferred embodiment, at least a portion of at least one of the two or more olefinic component streams, such as the third olefinic component stream 135 comprising C4+ olefins, can be passed to the OTO reaction zone 50 as olefinic co-feed stream 15 discussed above.

Where the olefinic product comprises ethylene, at least part of the ethylene may be further converted into at least one of polyethylene, mono-ethylene-glycol, ethylbenzene and styrene monomer. Where the olefinic product comprises propylene, at least part of the propylene may be further converted into at least one of polypropylene and propylene oxide.

The person skilled in the art will understand that the present invention can be carried out in many various ways without departing from the scope of the appended claims.

1. A process for the oxidative regeneration of a deactivated catalyst to provide a regenerated catalyst, said process comprising at least the steps of:
providing a catalyst comprising molecular sieve in hydrogen form to a guard zone;
passing a regeneration gas stream comprising oxidant through the guard zone to remove at least a part of one or both of any alkali metal ion and any alkaline earth metal ion from the regeneration gas stream by ion exchange with the catalyst, to provide a treated regeneration gas stream comprising oxidant;
providing deactivated catalyst comprising molecular sieve in hydrogen form to a regeneration zone, said deactivated catalyst obtained from one or both of an oxygenate to olefin process and an olefin cracking process, wherein said regeneration zone is separated from said guard zone such that there is no transfer of catalyst from the guard zone to the regeneration zone;
regenerating the deactivated catalyst in the regeneration zone with the treated regeneration gas stream to provide regenerated catalyst comprising regenerated molecular sieve in hydrogen form;
wherein said catalyst in said guard zone is one or both of deactivated catalyst comprising molecular sieve in hydrogen form comprising carbonaceous deposits and regenerated catalyst comprising regenerated molecular sieve in hydrogen form.

2. The process of claim 1 wherein the deactivated catalyst comprising molecular sieve in hydrogen form is provided by one or both of the steps:
reacting an oxygenate feedstock comprising oxygenate in an oxygenate reaction zone in the presence of a catalyst comprising molecular sieve in hydrogen form to produce the deactivated catalyst comprising molecular sieve in hydrogen form comprising carbonaceous deposits and a reaction effluent stream comprising unreacted oxygenate, olefinic product and water; and
reacting a C4+ hydrocarbon feedstock comprising olefin in an olefin cracking process reaction zone in the presence of a catalyst comprising molecular sieve in hydrogen form to produce the deactivated catalyst comprising molecular sieve in hydrogen form comprising carbonaceous deposits and a reaction effluent stream comprising unreacted C4+ hydrocarbon and an olefinic product comprising one or both of ethylene and propylene.

3. The process of claim 1 wherein the step of providing the catalyst comprising molecular sieve in hydrogen form to a guard zone comprises the step of:
passing a portion of either the deactivated catalyst from the regeneration zone or the regenerated catalyst from the regeneration zone to the guard zone.

4. The process of claim 1 wherein the regeneration gas stream comprises one or both of alkali metal ion and alkaline earth metal ion and said treated regeneration gas stream is depleted in one or more of alkali metal ion and alkaline earth metal ion.

5. The process of claim 1, wherein the step of passing a regeneration gas stream through the guard zone further provides spent catalyst comprising ion exchanged molecular sieve, said process further comprising the steps of:
removing spent catalyst comprising ion exchanged molecular sieve from the guard zone;
re-stocking the guard zone with catalyst comprising molecular sieve in hydrogen form selected from one or both of deactivated catalyst comprising molecular sieve in hydrogen form and regenerated catalyst comprising regenerated molecular sieve in hydrogen form.

6. The process of claim 1 wherein the guard zone and the regeneration zone are independently selected from a fixed bed, a fluid bed and a solid/gas contactor.

7. The process of claim 1 wherein the step of regenerating the spent catalyst comprises oxidising the carbonaceous deposits with the treated regenerated gas stream.

8. The process of claim 1 further comprising, prior to passing a regeneration gas stream through the guard zone, the step of:
heating a regeneration gas stream to provide a heated regeneration gas stream;
such that the treated regeneration gas stream is a heated stream.

9. The process of claim 1 further comprising, between the step of passing a regeneration gas stream through the guard zone and the step of regenerating the catalyst, the step of:
heating the treated regeneration gas stream to provide a treated regeneration gas stream as a heated stream.

10. The process of claim 1 wherein the oxidant comprises oxygen.

11. The process of claim 1 wherein the molecular sieve is selected from the group consisting of silicoaluminophosphate and aluminosilicate.

12. The process of claim 11 wherein the molecular sieve comprises one or more of the group consisting of a TON-type aluminosilicate, a MTT-type aluminosilicate, and a MFI-type aluminosilicate.

13. A process for the preparation of olefinic product, the process comprising at least the steps of:
(a) reacting an oxygenate feedstock comprising oxygenate in an oxygenate to olefin reaction zone in the presence of a OTO catalyst comprising molecular sieve in hydrogen form to produce a deactivated OTO catalyst comprising molecular sieve in hydrogen form comprising carbonaceous deposits and a reaction effluent stream comprising unreacted oxygenate, olefinic product and water;
(b) regenerating the deactivated OTO catalyst according to a process of claim 1, wherein the deactivated catalyst comprising molecular sieve in hydrogen form comprising carbonaceous deposits is the deactivated OTO catalyst, to provide a regenerated OTO catalyst.

14. A process for the preparation of olefinic product comprising one or both of ethylene and propylene, the process comprising at least the steps of:
(a) reacting an C4+ hydrocarbon feedstock comprising olefin in an olefin cracking process reaction zone in the presence of an OCP catalyst comprising molecular sieve in hydrogen form to produce a deactivated OCP catalyst comprising molecular sieve in hydrogen form and a reaction effluent stream comprising unreacted C4+ hydrocarbon and one or both of ethylene and propylene;
(b) regenerating the deactivated OCP catalyst according to a process of claim 1 to provide a regenerated catalyst, wherein the deactivated catalyst comprising molecular sieve in hydrogen form is the deactivated OCP catalyst.

15. The process of claim 13 further comprising the steps of:
(c) repeating step (a) at least once with the regenerated catalyst;
(d) repeating step (b) at least once with the deactivated catalyst.

16. An apparatus for the oxidative regeneration of a deactivated catalyst to provide a regenerated catalyst, said apparatus comprising at least:
a guard zone comprising one or both of deactivated catalyst comprising molecular sieve in hydrogen form and regenerated catalyst comprising regenerated molecular sieve in hydrogen form, said spent and regenerated catalyst from one or both of an oxygenate to olefin process and an olefin cracking process, said guard zone having a first inlet for a regeneration gas stream comprising oxidant and a first outlet for a treated regeneration gas stream comprising oxidant in fluid communication with a first inlet of a regeneration zone;
a regeneration zone comprising deactivated catalyst comprising molecular sieve in hydrogen form , said deactivated catalyst from one or both of an oxygenate to olefin process and an olefin cracking process, said regeneration zone having a first inlet for the treated regeneration gas stream and a first outlet for a regeneration effluent stream.
